(19) United States
(12) Patent Application Publication
(10) Pub. No.: US 2007/0036881 A1
(43) Pub. Date: Feb. 15, 2007

(54) Electrocoagulation and polymeric suspended solids reduction

(75) Inventor: Clark R. Griffith, Lansdale, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: MPC Inc., Wilmington, DE (US)

(21) Appl. No.: 11/203,396

(22) Filed: Aug. 12, 2005

(51) Int. Cl. C12P 7/06; C12C 11/00; C12M 3/00

(52) U.S. Cl. 426/11; 435/161; 435/289.1

(57) ABSTRACT

In the drying of thin stillage in a corn-to-ethanol process, suspended solids are largely separated from the thin stillage by electrocoagulation and polyacrylamide flocculation. Whole stillage contains solids that must be dried. Prior to the dryer, the whole stillage is processed in a centrifuge that generates wet cake. The wet cake goes directly to the dryer. The thin stillage from the centrifuge is treated with electrocoagulation and polyacrylamide polymer prior to being sent to a rotary screen drum and settling tank. The settling tank supernate is then sent to an evaporator. Condensate is taken off in the evaporator leaving syrup that is sent to the distiller's dried grains with solubles (DDGS) dryer along with the wet cake. The syrup is of significantly higher solids content with the current invention than with Prior Art technology. Energy savings in the DDGS dryer are achieved by the removal of water in the evaporation prior to drying

### FIELD OF THE INVENTION

This invention relates to a reduction of the amount of drying required of wet distiller's grains in a corn-to-ethanol production facility and more particularly to the use of electrocoagulation and polyacrylamide polymeric flocculation to reduce energy consumption in producing distiller's dried grains with solubles (DDGS).

### BACKGROUND OF THE INVENTION

The principal drive toward using ethanol as a gasoline additive is to more cleanly burn the fuel used in internal combustion engines. Additionally, with increasing depletion of economically recoverable petroleum reserves, the production of ethanol from vegetative sources as a replacement for conventional fossil-based liquid fuels becomes more attractive. In addition to offering promise as a practical and efficient fuel, biomass-derived ethanol in large quantities and at a competitive price has the potential for replacing certain petroleum-based chemical feedstocks. For example, ethanol can be catalytically dehydrated to ethylene, one of the most important of all chemical raw materials both in terms of quantity consumed and versatility in product synthesis.

U.S. Pat. No. 4,409,406, Feldman, is typical of a biomass derived ethanol process with extraction of water from the ethanol.

The article “Reducing Costs of Byproduct Recovery at Dry-Mill Ethanol Plants” by Robert C. Brown, of Iowa State University, www.eiorenew.iastate.edu is a study of the de-watering and drying issues when processing distiller dried grains into ethanol.

It is an object of the present invention to reduce energy costs in such processing.

### SUMMARY OF THE INVENTION

The object of the present invention is to reduce the amount of drying that takes place in the production of distiller's dried grains with solubles. This reduces auxiliary fuel consumption and reduces costs in corn-to-ethanol plants.

The slurry that remains after corn is fermented and ethanol and water are distilled off is called whole stillage (WStill). Whole stillage contains solids that need to be dried to 10% moisture in a dryer to be sold as distiller's dried grains (DDG) or distiller's dried grains with solubles (DDGS). Prior to the dryer the whole stillage is processed in a centrifuge that generates two streams, wet distiller's grains (WDG), or “wet cake” and the centrate, which is referred to as thin stillage (TStill). In accordance with the invention the suspended solids in the thin stillage are reduced by electrocoagulation and the addition of polyacrylamide polymers.

The foregoing and other objects, features and advantages of the invention will be better understood from the following more detailed description, drawings and appended claims.

### BRIEF DESCRIPTION OF THE DRAWINGS

FIG. 1 shows a prior art process;

FIG. 2 depicts the process of the present invention; and

FIG. 3 depicts the electro-coagulator in more detail.

### DETAILED DESCRIPTION OF PREFERRED EMBODIMENT

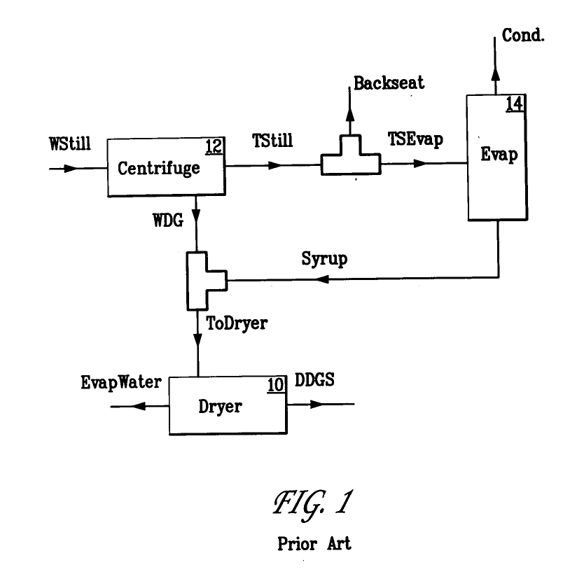

In the prior art process of FIG. 1 whole stillage (WStill) contains solids that need to be dried to 10% moisture in the dryer 10. Prior to the dryer, the wet stillage is processed in a decanter centrifuge 12 that generates two streams. One stream, wet distiller's grains (WDG), is wet cake (30-40% solids) that goes directly to the dryer 10. The other stream from centrifuge 12 is referred to as thin stillage which contains 5-10% total solids. Normally, the thin stillage goes directly to evaporator 14 for removal of water and conversion to “syrup” which contains 28-40% solids. The syrup is mixed with the wet cake from the centrifuge and sent to the dryer 10. The limitation on the evaporator's ability to increase the solids is largely dependent on the viscosity of the syrup, which increases with higher suspended solids in the thin stillage. Higher suspended solids increase evaporator tube fouling. The present invention reduces the suspended solids in the thin stillage so that the evaporator can increase the solid content of the syrup and reduce the evaporative load on the dryer 10.

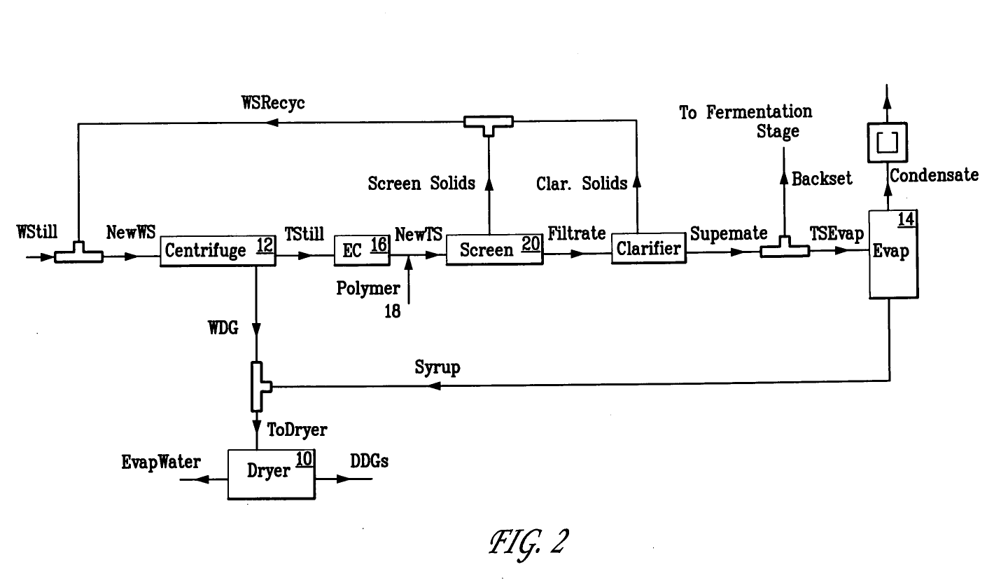

Referring to FIG. 2 an electro-coagulator 16 dissolves iron ions into the thin stillage. This, in part, adjusts the pH of the thin stillage without the addition of pH adjusting chemicals such as calcium oxide (lime), which might otherwise produce objectionable calcium deposits in the processing equipment and piping. It also adds iron ions that work with the polyacrylamide polymer to flocculate the suspended solids in the thin stillage. A source of polyacrylamide polymers 18 is blended with the thin stillage following the electro-coagulator 16. This causes the suspended solids to flocculate, making them easier to separate from the thin stillage.

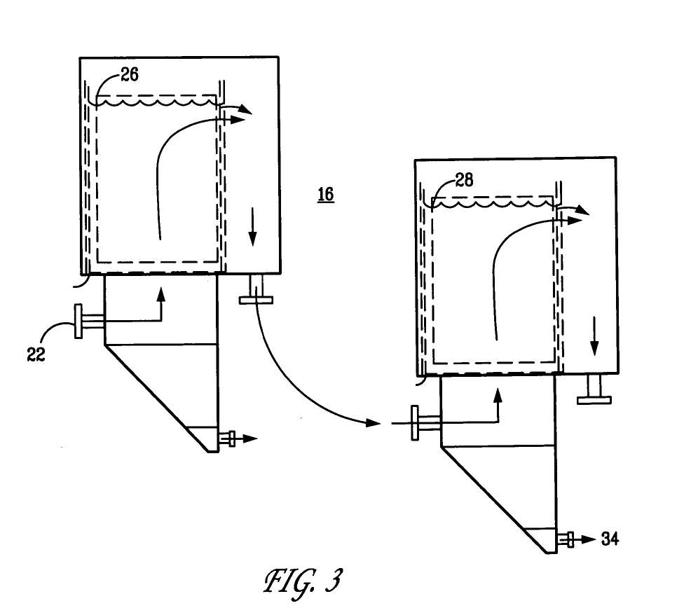

An electro-coagulator suitable for use is shown in FIG. 3 and is described more fully in http:\\www.kaselco.com, U.S. Pat. No. 5,928,493, Kaspar et al., and U.S. Patent Application 2004/0079650. Thin stillage is inputted at the inlet 22. The electro-coagulator includes parallel sacrifical metal plates 26 and 28 including thick plates and thin plates. The thick plates are electrically isolated from the thin plates. Power is applied to the thick plates and to the thin plates. The thin stillage flows through electro-coagulator 16 in an upward direction through the voids between the plates to an outlet 34. Addition of polyacrylamide polymer is performed subsequent to the outlet 34. The coagulation in the electro-coagulator and flocculation caused by the polymer allows solids in the new thin stillage to be easily removed.

Polyacrylamide has been used in waste water treatment, in making paper and in agriculture. See U.S. Pat. No. 5,942,086, Owen, U.S. Pat. No. 6,131,331, Duffy, Jr., and U.S. Pat. No. 6,632,774, Duffy, Jr., and U.S. Pat. No. 5,891,254, Coville et al.

Commonly used polyacrylamides are random copolymers of the monomers acrylic acid and acrylamide:

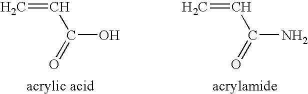

A typical anionic flocculant is a copolymer of acrylamide and acrylic acid and is made by inverse emulsion polymerization.

Polyacrylamides have been found to be safe for ingestion by animals. This makes polyacrylamide particularly useful in the process stream of the present invention wherein solid by products are frequently used for animal feed.

Polyacrylamides suitable for use are available from Met-Pro Corporation, Harleysville, Pa. Compositions which are suitable for use are 30-35 mole-percent charge, high and very high molecular weight anionic polyacrylamide polymers in a water-oil emulsion and anionic dry polymer of similar charge and molecular weight

Information on a suitable polyacrylamide follows:

OSHA REGULATED COMPONENTS

| Component/CAS No. | % (w/w) | OSHA (PEL): |
|---|---|---|
| Petroleum distillate | 20.5-22.5 | 500 ppm |
| hydrotreated light 64742-47-8 | | 1200 mg/m³ (Supplier) |
| | | 155 ppm (Supplier) |
| Alcohols (C10-16), | 2.3-2.7 | Not Established |
| ethoxylated 68002-97-1 | | |

PHYSICAL AND CHEMICAL PROPERTIES

| | |
|---|---|
| Color: | grayish-white |
| Appearance: | emulsion |
| Odor: | ammonia |
| Boiling Point: | ˜177-260° C. 350-500° F. |
| Melting Point: | −18° C. −0° F. |
| Specific Gravity: | ˜1.0 |
| Percent Volatile (% by wt): | 64-65 |
| pH | 6.0-8.0 in water |
| Solubility in Water: | Limited by viscosity |
| Volatile Organic Content: | 22% (g/g) |
| Flash Point: | >93° C. 200° F. Closed Cup |

The coagulation mechanism of iron ions is charge neutralization. Particles with like charge repel one another. Removal of the charge enables particles to approach close enough to coagulate using polyacrylamide polymers. This coagulation results from polymer chains bridging particles to create larger masses that settle out and are large enough to be removed. Both cationic polymers and anionic polymers can be used in different circumstances.

Referring again to FIG. 2, the flocculated solids are removed with a rotary screen 20. The rotary screen is a horizontal cylinder. The flocculated thin stillage flows onto the outside of the screen drum. The majority of the liquids go through the screen. The majority of the flocculated solids remain on the outside of the screen drum. The liquids that penetrate the screen (Filtrate) are sent to clarifier 21 which is a settling tank. The solids (Screen Solids) are combined with Clar. Solids from settling tank 21 to form WSRecyc. This is pumped back to the inlet of centrifuge 12 for further de-watering.

The supernatant liquids (Supernate) from the settling tank are pumped to evaporator 14 for further de-watering. The evaporator produces a condensate stream and the solids are concentrated into a fluid called “syrup.” The syrup solids can be increased to 66.5% while the viscosity of the syrup approximately equals typical syrup at 30-40% solids.

Another benefit of the invention includes altering the settings of the centrifuge 12 to produce drier wet cake (WDG). This further reduces the evaporative loading of dryer 10. Also, a portion of the thin stillage called “backset” is sent to the fermentation stage of the ethanol production process. Reducing the level of suspended solids in the backset allows for additional corn solids to be added to the fermentation stage of the corn-to-ethanol process. This increases ethanol production.

### EXAMPLES

The following tables list exemplary process parameters for operation of the process in accordance with the with the prior art and in accordance with the present invention.

PRIOR ART

| | | | |
|---|---|---|---|
| Volume of Whole Stillage into Centrifuge 12 (WStill) | 311379 | lb/hr | (% solids) |
| | 561 | gal/min 180° F. | 12.5 |
| Volume of Thin Stillage from Centrifuge 12 (TStill) | 239521 | lb/hr | 6.5 |
| Flow of Wet Dried Grain from Centrifuge 12 (WDG) | 71859 | lb/hr | 32.5 |
| Flow to Dryer 10 (To Dryer) | 115649 | lb/hr | 30.8 |
| Flow of Evaporated Water from Dryer 10 (Evap Water | 76072 | lb/hr | 0.0 |
| Flow of Distiller Dried Grains with Solubles from | 39576 | lb/hr | 90.0 |
| Dryer 10 (DDGS) | | | |
| Flow of BackSet (Backset) | 50778 | lb/hr | 6.5 |
| Flow of Thin Stillage Evaporated (TSEvap) | 188742 | lb/hr | 6.5 |
| Flow of Condensate from Evaporator 14 (Cond) | 298 | gal/min | 0.0 |
| | 144852 | lb/hr | |
| Flow of Syrup from Evaporator (Syrup) | 65 | gal/min | 28.1 |
| Energy Required By Dryer 10 | 95,100,000 | Btu/hr | N/A |

In an exemplary embodiment of the invention, the thin stillage was treated in an electro-coagulator obtained from Kaspar Electroplating Corporation, assignee of the aforementioned Kaspar et al. patent and patent application. Subsequently a 30 mole-percent charged high molecular weight polyacrylamide polymer was added to it. Using data from this testing the Prior Art material balance can be adjusted producing the following material balance. This material balance incorporates the simplifying assumption that the incoming whole stillage flow rate and percent solids is identical to the Prior Art material balance.

| Volume of Whole Stillage into Centrifuge 12 (WStill) | 311379 | lb/hr | % Solids |
|---|---|---|---|
| | 561 | gal/min 180° F. | 12.5 |
| Volume of Thin Stillage from Centrifuge 12 (TStill) | 321449 | lb/hr | 7.4 |
| Flow of Wet Dried Grain from Centrifuge 12 (WDG) | 84076 | lb/hr | 23.0 |
| Flow to Dryer 10 (To Dryer) | 102087 | lb/hr | 37.0 |
| Flow of Evaporated Water from Dryer 10 (EvapWater) | 60248 | lb/hr | 0.0 |
| Flow of Distiller Dried Grains with Solubles from | 41838 | | 90.3 |
| Dryer 10 (DDGS) | | | |
| Flow of Backset (Backset) | 24000 | | 4.8 |
| Flow of Thin Stillage Evaporated (TSEvap) | 209989 | | 4.8 |
| Flow of Condensate from Evaporator 14 (Cond | 191987 | | 0.0 |
| Flow of Syrup from Evaporator (Syrup) | 18011 | | 55.6 |
| Flow of polyacrylamide polymer from Met-Pro | 6662 | lb/hr | N/A |
| Corporation (Polymer) | | | |
| Energy Required By Dryer 10 | 75,300,000 | Btu/hr | N/A |

It has been found that the ion addition of the electro-coagulator together with the polyacrylamide flocculants provide particularly efficient energy reduction.

It will be understood that various modifications to the process can be made. The appended claims cover all such modifications within the true spirit and scope of the invention.

What is claimed:

1. A method of removing suspended solids from a process stream in ethanol production comprising:
adding polyacrylamide polymer to said process stream to promote said separation.

2. The method recited in claim 1 further comprising:
passing said process stream through an electro-coagulator device prior to adding said polymer.

3. The method recited in claim 1 when said process stream is stillage.

4. The method recited in claim 3 wherein said stillage is thin stillage in a corn-to-ethanol process.

5. The method recited in claim 4 further comprising drying said stillage and wherein said adding of polymers and said electrocoagulation is performed prior to drying to conserve energy required in said drying.

6. The method recited in claim 1 wherein said polyacrylamide polymer is one of an anionic polyacrylamide in a water in oil emulsion or a dry polymer suspended in water.

7. The method recited in claim 2 wherein said electro-coagulator has multiple flat opposing sacrificial plates.

8. The method recited in claim 7 wherein said process stream flows in an upward flow path between said plates.

9. The method recited in claim 7 wherein said plates are oppositely charged electrically.

10. Apparatus for removing suspended solids from a process stream in ethanol production comprising;
an electro-coagulator, said process stream passing through said electro-coagulator;
and a dryer, said process stream being applied to said dryer after passing through said electro-coagulator.

11. The apparatus recited in claim 10 further comprising;
means for adding polyacryilamide polymer to said process stream.

12. The apparatus recited in claim 10 further comprising;
a centrifuge for removing solids from said process stream prior to applying said process stream to said electro-coagulator.

13. The apparatus recited in claim 12 further comprising;
an evaporator, said process stream being applied to said evaporator after treatment in said electro-coagulator.

14. The apparatus recited in claim 12 further comprising;
a rotary screen drum, the process stream from said electro-coagulator being applied to said screen drum to separate solids from liquid.

15. The apparatus recited in claim 14 wherein said solids are recirculated to said dryer.

16. The apparatus recited in claim 14 further comprising;
a settling tank, the liquid process stream from said rotary screen drum entering said settling tank to further separate solids from liquids.

17. The apparatus recited in claim 16 wherein said solids from said process stream prior to applying said process stream to said electro-coagulator wherein said solids from said rotary screen drum and solids from said settling tank are re-circulated to said centrifuge.

18. The apparatus recited in claim 14 wherein a portion of said process stream containing polymer is returned to said ethanol production stage.
